(19) United States
(12) Patent Application Publication
(10) Pub. No.: US 2026/0306798 A1
(43) Pub. Date: Oct. 8, 2026

(54) INTRAVENOUS LINE SECURING DEVICE

(71) Applicant: Ronald Yerdon, Parish, NY (US)

(72) Inventor: Ronald Yerdon, Parish, NY (US)

(21) Appl. No.: 19/097,973

(22) Filed: Apr. 2, 2025

(51) Int. Cl. A61M 25/02; A61M 5/14

(52) U.S. Cl. CPC A61M 25/02; A61M 5/1413; A61M 2025/0206

(57) ABSTRACT

An intravenous line securing device includes a panel having a front side. A shaft extends from the front side of the panel. A hook extends from the front side of the panel spaced from the shaft to receive and engage an intravenous line extending out from a port site, around the shaft, and back to the hook to secure the intravenous line to the panel. An outer sleeve is elastic for positioning on an arm and over the panel to inhibit movement of the panel on the arm.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention.

The disclosure relates to intravenous line securing device and more particularly pertains to a new intravenous line securing device for securing an intravenous line to prevent discomfort and inadvertent disconnection of the intravenous line from a port site.

### (2) Description of Related Art Including Information Disclosed Under 37 CFR 1.97 and 1.98.

The prior art relates to intravenous line securing device. Known prior art lacks engagement and securing of an intravenous line in a quickly accessible yet stable position.

### BRIEF SUMMARY OF THE INVENTION

An embodiment of the disclosure meets the needs presented above by generally comprising a panel having a front side. A shaft extends from the front side of the panel. A hook extends from the front side of the panel spaced from the shaft to receive and engage an intravenous line extending out from a port site, around the shaft, and back to the hook to secure the intravenous line to the panel. An outer sleeve is elastic for positioning on an arm and over the panel to inhibit movement of the panel on the arm.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top front side perspective view of an intravenous line securing device according to an embodiment of the disclosure.

FIG. 2 is a front view of an embodiment of the disclosure.

FIG. 3 is a side view of an embodiment of the disclosure.

FIG. 4 is a front view of an embodiment of the disclosure in use.

FIG. 5 is a front view of an embodiment of the disclosure in use.

FIG. 6 is a front view of an embodiment of the disclosure in use.

### DETAILED DESCRIPTION OF THE INVENTION

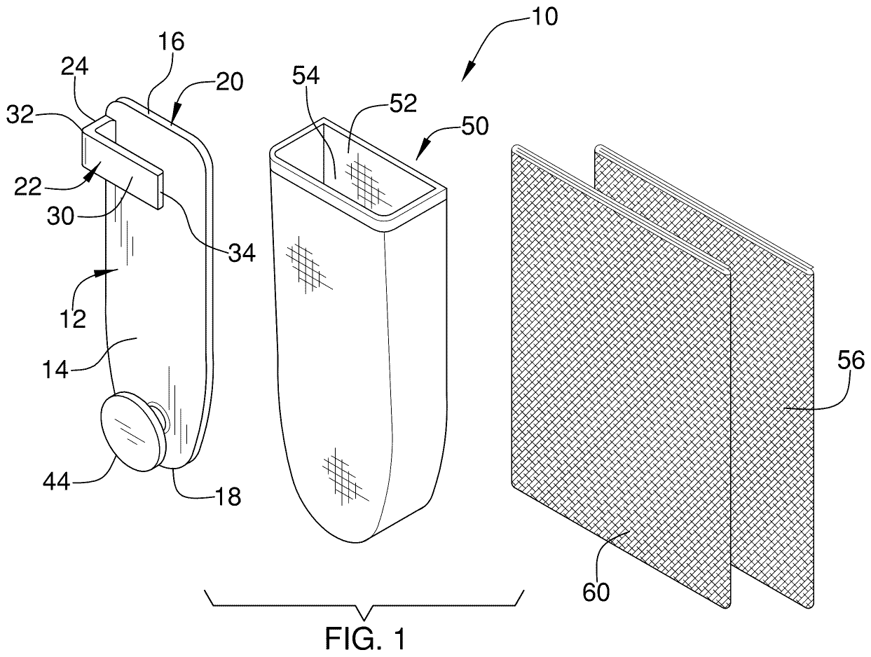

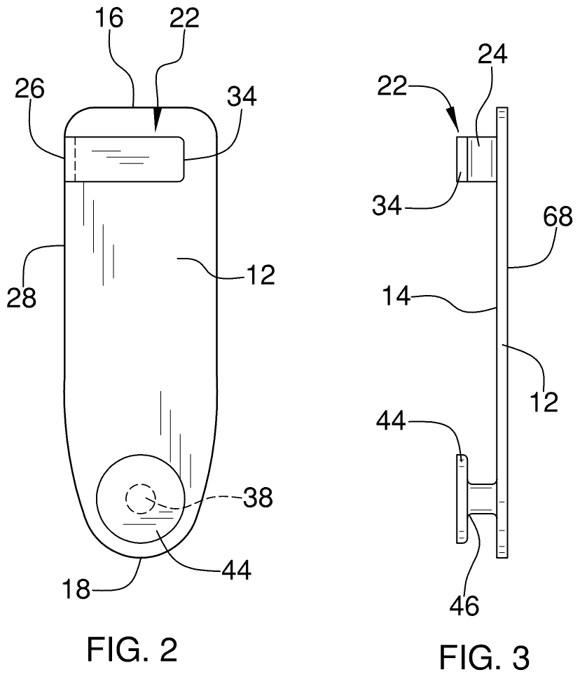

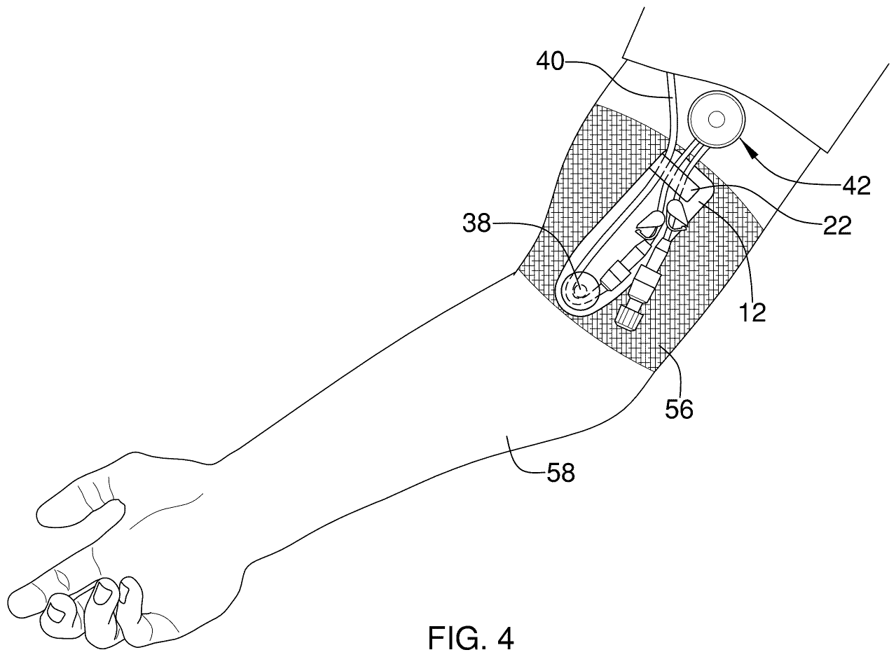

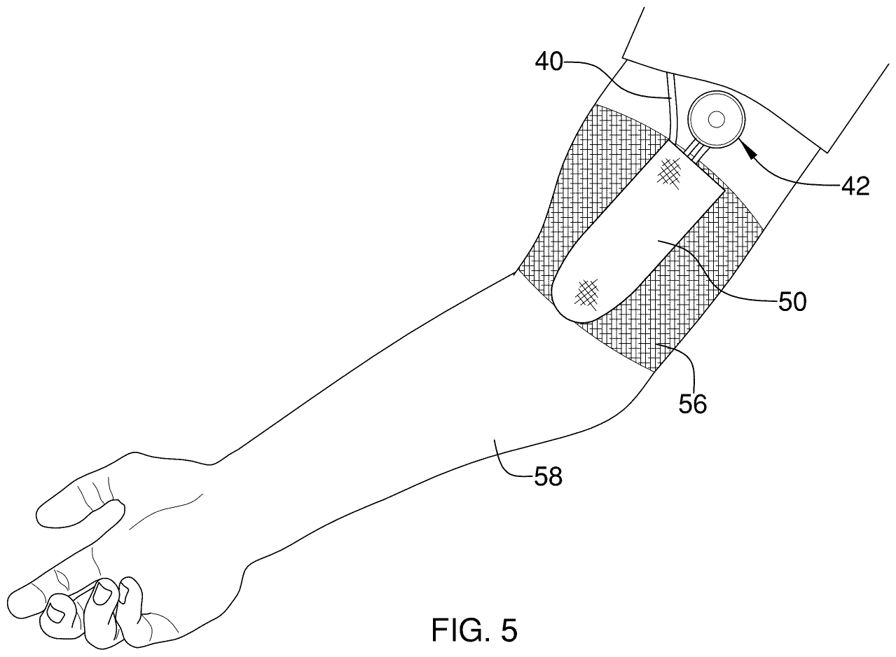

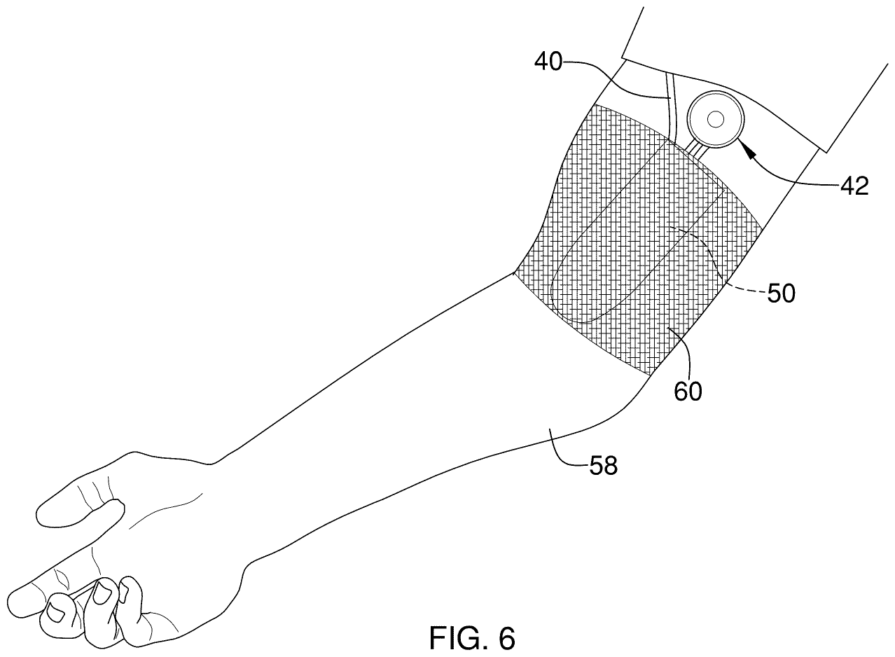

With reference now to the drawings, and in particular to FIGS. 1 through 6 thereof, a new intravenous line securing device embodying the principles and concepts of an embodiment of the disclosure and generally designated by the reference numeral 10 will be described.

As best illustrated in FIGS. 1 through 6, the intravenous line securing device 10 generally comprises a panel 12 having a front side 14. The panel 12 is elongated between a first end 16 and a second end 18. The first end 16 is a straight edge 20 and the second end 18 is rounded. A hook 22 extends from the front side 14 of the panel 12. The hook 22 is positioned proximate to the first end 16 the hook 22 is elongated and parallel to the straight edge 20. The hook 22 has a first flange 24 coupled to and extending from the front side 14 of the panel 12. The first flange 24 has an outer face 26 positioned flush with a peripheral edge 28 of the front side 14 of the panel 12. The hook 22 has a second flange 30 coupled to and extending from a distal end 32 of the first flange 24 relative to the panel 12. The second flange 30 has a length less than a width of the panel 12 wherein a free end 34 of the second flange 30 is inset relative to the peripheral edge 28 of the front side 14 of the panel 12. A shaft 38 extends from the front side 14 of the panel 12. The hook 22 is spaced from the shaft 38 wherein the shaft 38 is configured to receive an intravenous line 40 extending around the shaft 38 with the hook 22 engaging the intravenous line 40 such that the intravenous line 40 is secured to the panel 12. The shaft 38 is positioned proximate to the second end 18. The intravenous line 40 can be extended through the hook 22 adjacent to a port site 42, then extended around the shaft 38, and then back through the hook 22. A disc 44 is positioned at a distal end 46 of the shaft 38 relative to the panel 12. An outer diameter of the disc 44 is greater than a diameter of the shaft 38 wherein the disc 44 is configured for holding the intravenous line 40 adjacent to the panel 12 when the intravenous line 40 is extended around the shaft 38. A sheath 50 has an open end 52 into an interior space 54. The panel 12 is positionable in the interior space 54 of the sheath 50 such that the open end 52 of the sheath 50 is configured for the intravenous line 40 to extend into and out of the interior space 54 when the intravenous line 40 is secured to the panel 12.

An inner sleeve 56 is elastic. The inner sleeve 56 may be of the resiliently stretchable tubular kind for positioning on an arm 58 adjacent to the port site 42. The inner sleeve 56 is positioned under the panel 12. A width of the inner sleeve 56 is at least a length of the panel 12 so the an entirety of a back side 68 of the panel 12 can be positioned on the inner sleeve 56. An outer sleeve 60, which may be of the same type and construction as the inner sleeve 56, is elastic such that the outer sleeve 60 is positionable on the arm 58 and over the panel 12 within the sheath 50. Thus, the outer sleeve 60 inhibits movement of the panel 12 on the arm 58. A width of the outer sleeve 60 is also at least a length of the panel 12.

In use, the inner sleeve 56 is positioned on the arm 58 adjacent to the port site 42. The intravenous line 40 is attached to the panel 12 as described above. The sheath 50 is then place over the panel 12 and portion of the intravenous line 40 engaged by the hook 22 and shaft 38. The outer sleeve 60 is then positioned over the sheath 50 and panel 12 to secure them in place adjacent to the port site 42.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An intravenous line securing device comprising:
a panel having a front side;
a shaft extending from said front side of said panel;
a hook extending from said front side of said panel, said hook being spaced from said shaft wherein said hook is configured to receive and engage an intravenous line extending out from a port site and around said shaft and back to said hook wherein such that the intravenous line is secured to said panel; and
an outer sleeve, said outer sleeve being elastic such that said outer sleeve is configured for positioning on an arm and over said panel whereby said outer sleeve inhibits movement of the panel on the arm.

2. The intravenous line securing device of claim 1, further comprising a disc positioned at a distal end of said shaft relative to said panel, an outer diameter of said disc being greater than a diameter of said shaft wherein said disc is configured for holding the intravenous line adjacent to said panel when the intravenous line is extended around said shaft.

3. The intravenous line securing device of claim 1, further comprising said panel being elongated between a first end and a second end.

4. The intravenous line securing device of claim 3, further comprising said hook being positioned proximate to said first end and said shaft being positioned proximate to said second end.

5. The intravenous line securing device of claim 4, further comprising said first end being a straight edge and said second end being rounded, said hook being elongated and parallel to said straight edge.

6. The intravenous line securing device of claim 1, further comprising said hook having a first flange coupled to and extending from said front side of said panel, said first flange having an outer face positioned flush with a peripheral edge of said front side of said panel.

7. The intravenous line securing device of claim 6, further comprising said hook having a second flange coupled to and extending from a distal end of said first flange relative to said panel, said second flange having a length less than a width of said panel wherein a free end of said second flange is inset relative to said peripheral edge of said front side of said panel.

8. The intravenous line securing device of claim 1, further comprising a sheath having an open end into an interior space, said panel being positionable in said interior space of said sheath such that the open end of the sheath is configured for the intravenous line to extend into and out of the interior space when the intravenous line is secured to the panel.

9. The intravenous line securing device of claim 1, further comprising an inner sleeve, said inner sleeve being elastic wherein said inner sleeve is configured for positioning on the arm and under the panel.

10. The intravenous line securing device of claim 1, wherein a width of said outer sleeve is at least a length of said panel.

11. The intravenous line securing device of claim 9, wherein a width of said inner sleeve is at least a length of said panel.

12. An intravenous line securing device comprising:
a panel having a front side, said panel being elongated between a first end and a second end, said first end being a straight edge and said second end being rounded;
a hook extending from said front side of said panel, said hook being positioned proximate to said first end, said hook being elongated and parallel to said straight edge, said hook having a first flange coupled to and extending from said front side of said panel, said first flange having an outer face positioned flush with a peripheral edge of said front side of said panel, said hook having a second flange coupled to and extending from a distal end of said first flange relative to said panel, said second flange having a length less than a width of said panel wherein a free end of said second flange is inset relative to said peripheral edge of said front side of said panel;
a shaft extending from said front side of said panel, said hook being spaced from said shaft wherein said shaft is configured to receive an intravenous line extending around said shaft with said hook engaging the intravenous line such that the intravenous line is secured to said panel, said shaft being positioned proximate to said second end;
a disc positioned at a distal end of said shaft relative to said panel, an outer diameter of said disc being greater than a diameter of said shaft wherein said disc is configured for holding the intravenous line adjacent to said panel when the intravenous line is extended around said shaft;
a sheath having an open end into an interior space, said panel being positionable in said interior space of said sheath such that the open end of the sheath is configured for the intravenous line to extend into and out of the interior space when the intravenous line is secured to the panel;
an inner sleeve, said inner sleeve being elastic wherein said inner sleeve is configured for positioning on the arm and under the panel, wherein a width of said inner sleeve is at least a length of said panel; and
an outer sleeve, said outer sleeve being elastic such that said outer sleeve is configured for positioning on an arm and over said panel whereby said outer sleeve inhibits movement of the panel on the arm, wherein a width of said outer sleeve is at least a length of said panel.
